(19) United States
(12) Patent Application Publication
(10) Pub. No.: US 2011/0195996 A1
(43) Pub. Date: Aug. 11, 2011

(54) Transmucosal Treatment Methods in Patients With Mucositis

(75) Inventors: Mona Darwish, Malvern, PA (US); Philmore Robertson, JR., West Chester, PA (US)

(73) Assignee: CEPHALON, INC., Frazer, PA (US)

(21) Appl. No.: 13/089,578

(22) Filed: Apr. 19, 2011

Related U.S. Application Data
(63) Continuation of application No. 12/150,055, filed on Apr. 24, 2008, now abandoned.
(60) Provisional application No. 60/925,981, filed on Apr. 24, 2007.

(51) Int. Cl. A61K 31/4468; A61P 29/00

(52) U.S. Cl. 514/329

(57) ABSTRACT

In accordance with the invention is an fentanyl-containing dosage form designed for transmucosal delivery which includes both an effervescent couple and a pH adjusting substance which can be administered to patients suffering from both pain and mucositis. Also provided is a method of treating pain in a patient having mild oral mucositis by administering a fentanyl-containing dosage form designed for transmucosal delivery.

### BACKGROUND OF THE INVENTION

Fentanyl (CAS Registry No. 437-38-7) and its salts are opioids, controlled substances, and extremely potent narcotic analgesics. Fentanyl and its citrate salt are currently marketed by a number of companies in a number of delivery formats. Fentanyl was introduced into medical practice in the 1960s as an intravenous anesthetic under the trade name Sublimaze. Fentanyl has an LD₅₀ of 3.1 mg/kg in rats, and 0.03 mg/kg in monkeys. The LD₅₀ in humans is not known.

Fentanyl is also available as an injectable, a transdermal patch (such as Duragesic® by Janssen Pharmaceutica), and an oral lozenge on a stick (such as ACTIQ®, available from Cephalon, Inc.). As to the lozenge, three patents were identified in past editions of the FDA publication Approved Drug Products With Therapeutic Equivalence Evaluations (hereinafter “the Orange Book”) as relating to ACTIQ®: U.S. Pat. Nos. 4,671,953, 4,863,737 and 5,785,989.

ACTIQ®, a flavored lozenge on a stick, is swabbed over the mucosal surfaces inside the mouth to enable delivery through the oral mucosa. ACTIQ® is indicated for opioid-tolerant patients and is effective in treating breakthrough cancer pain. In clinical trials of ACTIQ®, breakthrough cancer pain was defined as a transient flare of moderate-to-severe pain occurring in cancer patients experiencing persistent cancer pain otherwise controlled with maintenance doses of opiate medications, including at least 60 mg of morphine/day, 50 μg transdermal fentanyl/hour or equal analgesic dose of another opiate for a week or longer.

U.S. Patent Publication Nos. 2005/0163838A1, 2005/0169989A1 and 2005/0142197A1, owned by CIMA LABS, INC., a Cephalon Company, describe effervescent oral opiate dosage forms that include substantially less opiate by weight as compared with other known oral formulations including ACTIQ®. The dissolved oral opiate dosage forms disclosed are intended for oral administration across the oral mucosa. Products falling within the scope of at least some of these applications, and others as described herein, are currently marketed under the trademark FENTORA® fentanyl buccal tablets (“FBT”). FENTORA® is indicated for the management of breakthrough pain in patients with cancer who are already receiving and who are tolerant to opioid therapy for their underlying persistent pain. One advantage of FENTORA®, according to the prescribing information, is that Fentora® has faster absorption of the API into the bloodstream and lower dosage levels.

U.S. Pat. No. 6,200,604, which issued Mar. 13, 2001 to CIMA LABS INC., a Cephalon Company, exemplifies two fentanyl formulations each containing 36% effervescence and 1.57 mg of fentanyl salt. See example I thereof, column 5, line 60 through column 6, line 30. The '604 Patent describes the use of, amongst other things, effervescence as a penetration enhancer for influencing oral drug absorption. See also U.S. Pat. Nos. 6,759,059 and 6,680,071. See also Brendenberg, S., 2003 New Concepts in Administration of Drugs in Tablet Form: Formulation and Evaluation of a Sublingual Tablet for Rapid Absorption, and Presentation of an Individualized Dose Administration System, Acta Universitiatis Upsaliensis. Comprehensive Summaries of Uppsala Dissertations from the Faculty of Pharmacy, 287, 83 pp. Uppsala ISBN 91-554-5600-6.

The tolerability and safety of two different formulations of oral transmucosal fentanyl citrate (“OTFC”) were tested in cancer patients having grades 3 and 4 radiation-induced oral mucositis (see Lauren Shaiova, “Tolerability and Effects of Two Formulations of Oral Transmucosal Fentanyl Citrate (OTFC; ACTIQ) in Patients with Radiation-Induced Oral Mucositis,” Support Care Cancer (2004) 12:268-273). The study suggested that the two different fentanyl formulations were well tolerated and that the treatments produced progressively less oral mucositis pain after administration.

Pain is a prevalent symptom in cancer patients, affecting up to 50% of patients undergoing active cancer treatment and up to 90% of those with advanced disease. In these cancer populations, over half of those with chronic pain on opioid therapy will experience breakthrough pain, defined as a transient exacerbation of pain that occurs with otherwise stable, controlled, persistent pain. Providing pain relief from breakthrough pain is inexorably linked with the patient's immediate quality of life. And for terminal cancer patients, providing pain relief may be the only thing that medical science can offer.

Unfortunately, there is a subpopulation of patients in need of pain relief through opiate therapy that also suffer from a condition called mucositis. Oral mucositis is an acute, painful, and sometimes debilitating complication of cancer surgery, chemotherapy and radiation. It occurs in 20-40% of patients treated with chemotherapy alone and up to 50% of patients receiving combination radiation and chemotherapy, especially those with head and neck cancer. Mucositis occurs when cancer treatments break down the rapidly divided epithelial cells lining the gastrointestinal tract, particularly in the oral cavity, leaving the mucosal tissue open to ulceration and infection. The consequences of mucositis can be mild requiring little intervention to severe (hypovolemia, electrolyte abnormalities, and malnutrition) that may result in fatal complications.

It was believed by the inventors that patients suffering from mild oral mucositis, generally designated as mucositis grade 2 or less, would require caution when dosing fentanyl transmucosally, because mucositis would affect drug absorption and/or flux across the oral mucosa. This could manifest itself in a clinically significant increase in the bioavailability of the drug, increasing the initial blood concentration which could expose the patient to unsafe concentrations. As such, those patients were administrated pain treatments through other methods, such as injection, increasing the suffering of those already coping with unbearable amounts of pain.

Ideally, every dosing option should be made available to those patients suffering from pain such that they can use which ever treatment best suits their needs. For terminal cancer patients, in particular, anything that can improve their quality of life is highly desirable. Certainly, transmucosal administration would be one convenient, comfortable, and relatively pain-free method of administration for those with mucositis if it were possible.

### SUMMARY OF THE INVENTION

One aspect of the invention is a fentanyl-containing dosage form designed for oral transmucosal delivery which includes both an effervescent couple and a pH adjusting substance and which can be administered to patients suffering from both pain and oral mucositis. In some embodiments, the size, shape, composition, and/or physical properties of the dosage form can be adjusted to provide a dosage form particularly well suited for patients with mild oral mucosities, even when compared to other oral effervescent fentanyl formulations.

Another aspect of the invention are methods of treating pain comprising administering to a patient in need thereof and who has oral mucositis, a dosage form comprising fentanyl (in free base form or a salt thereof). The dosage form is designed for the administration of fentanyl across the oral mucosa through buccal, gingival or sublingual administration routes. The dosage form is placed in intimate contact with the oral mucosa and retained in intimate contact with the oral mucosa, for a time sufficient to allow uptake of at least a therapeutically significant amount of fentanyl across the oral mucosa. The dosage form comprises an amount of fentanyl sufficient to treat the patient's pain, generally between about 50 μg to 1400 μg per dosage form, at least one pH adjusting substance, and at least one effervescent couple. No clinically significant change in fentanyl uptake is realized when these formulations are dosed in patients having mild oral mucositis as compared with dosing the same dosage forms in similar patients not having mucositis. In another aspect, dosing in patients having mucositis increases fentanyl uptake by no more than and about 15% as compared with fentanyl uptake in patients not having mucositis.

In another aspect, the amount of fentanyl ranges from about 100 μg to about 1250 μg by weight of the dosage form. In another aspect, the amount of fentanyl ranges from about 100 μg to about 1000 μg by weight of the dosage form.

In the methods of the invention, the doses to be administered are also from about 50 μg to about 1400 μg per dose, which may be given in one dosage form or divided into a number of dosage forms.

In another aspect, the method further comprises the steps of ingesting a liquid such that the patient's mouth becomes at least partially filled with the liquid and swallowing the liquid and any of the dosage form remaining in the mouth.

In another aspect, the oral dosage form is placed and retained between an upper gum and a cheek (buccal administration). In another aspect, the oral dosage form is placed and retained under the tongue (sublingual).

In another aspect, the dosage form is maintained in intimate contact with the oral mucosa, preferably with a minimum of movement, for between about 5 and about 30 minutes. In still another aspect, the surface area of the dosage form ranges from about 10=² to about 160=².

### BRIEF DESCRIPTION OF THE DRAWINGS

FIG. 1 is a graph of the mean plasma concentrations of fentanyl following administration of FENTORA® in opioid-tolerant cancer patients with or without mucositis.

### DETAILED DESCRIPTION

The inventors believed that the tolerability and intramucosal absorption of an oral composition of fentanyl, such as FENTORA®, would be clinically significantly different in patients having mild oral mucositis compared with those patients not having oral mucositis. Specifically, it was believed that mild oral mucositis would cause at least about a 25% increase in fentanyl uptake, as measured by Cₘₐₓ (the maximum blood concentration), an increase which would be considered clinically significant. As used herein, the term “uptake” means the transfer of fentanyl across the oral mucosa, generally followed by subsequent release into systemic circulation. The inventors opined that the local tissue damage and/or the inflammation associated with mucositis would offer less resistance to absorption of fentanyl across the oral mucosa. It was believed that this lowered resistance to absorption would result in higher than expected Cₘₐₓ values and could trigger fentanyl induced side-effects. This could necessitate reducing the dose for those having mucositis as compared with those not having mucositis. While such a change could reduce the chance and/or severity of the possible side effects, it could also reduce the level of pain relief.

It has been found, unexpectedly, that the condition of mild oral mucositis does not cause a clinically significant change in fentanyl uptake from dosage forms as described herein. By “does not cause a clinically significant change” or “no clinically significant change” (used interchangeably) it is meant that any increase in fentanyl uptake through the oral muscosa, measured by comparing the maximum plasma concentrations (Cₘₐₓ) of fentanyl in patients having mucositis with patients not having mucositis, is at most about 20% greater for those patients having mucositis. Preferably, when dosed using the dosage forms of the invention, patients having mucositis experience an increase in uptake of not more than about 15%, as compared with those patients not having musocitis. As such, it has been discovered that when patients having mild oral mucositis are dosed with FENTORA®, no change in dosage strength or regimen is needed as compared with those not having mucositis.

Moreover, it has been found that the maximum plasma concentrations were achieved rapidly in patients with and without oral mucositis, suggesting that the condition of mild oral mucositis has a minimal effect on the absorption of fentanyl across the oral mucosa from these dosage forms. Moreover, when certain physical characteristics of the dosage forms are controlled, it may also be possible to obtain beneficial results.

The present invention includes, in one aspect, a method of treating pain comprising administering to a patient having mild oral mucositis (i.e., clinical grade 1 oral mucositis) a dosage form comprising fentanyl, the dosage form exhibiting no clinically significant change in fentanyl uptake when dosed in patients having mild oral mucositis as compared with those patients not having mucositis.

This method of treatment comprises the steps of contacting the oral mucosa of a patient having mild oral mucositis with an oral dosage form designed for the administration of fentanyl across the oral mucosa through buccal, gingival, or sublingual administration routes. The oral dosage form is placed and retained in intimate contact with the oral mucosa so as to facilitate transport of a therapeutically significant amount of fentanyl through the oral mucosa and into the bloodstream. As used herein, the term “therapeutically significant amount” refers to an amount of fentanyl that is effective to treat a patient's pain. In some embodiments, the dosage form is placed between an upper or lower gum and a cheek so as to facilitate buccal or gingival administration. In other embodiments, the dosage form is placed under the tongue so as to facilitate sublingual administration. Regardless of where the dosage form is placed, it is preferred that the dosage form be retained in place with minimal movement.

Typically, the dosage form is kept in contact with the oral mucosa for up to about 30 minutes, or until substantially all of the dosage form dissolves or disintegrates. In preferred embodiments, the dosage form is kept in contact with the oral mucosa for between about 5 minutes and about 30 minutes.

The amount of fentanyl contained in the dosage form depends on the treatment level sought, the patient, the patient's condition, and the sound judgment of the medical professionals involved. As used herein, the term “fentanyl” refers to fentanyl free-base and the salt forms thereof including, but not limited to, the citrate salt, the hydrochloride salt, the tartaric acid salt, and the succinic acid salt. To be effective, the fentanyl-containing dosage form should contain sufficient fentanyl to treat the patient's pain. In general, the amount of fentanyl ranges between about 0.01% to about 5% by weight of the dosage form, preferably ranging from about 0.05% to about 3% by weight of the dosage form.

The amount of fentanyl, based on the weight of the free-base of fentanyl or an equivalent amount of a fentanyl salt, in the dosage form may range from about 50 μg to about 1400 μg, preferably from about 100 μg to about 1250 μg, more preferably from about 100 μg to about 1000 μg. In one embodiment, the amount of fentanyl is 50 μg. In another embodiment, the amount of fentanyl is 100 μg. In a further embodiment, the amount of fentanyl is 150 μg. In yet another embodiment, the amount of fentanyl is 200 μg. In yet another embodiment, the amount of fentanyl is 300 μg. In yet another embodiment, the amount of fentanyl is 400 μg. In yet another embodiment, the amount of fentanyl is 500 μg. In yet another embodiment, the amount of fentanyl is 600 μg. In yet another embodiment, the amount of fentanyl is 800 μg. In yet another embodiment, the amount of fentanyl is 1000 μg. In yet another embodiment, the amount of fentanyl is 1250 μg. In yet another embodiment, the amount of fentanyl is 1400 μg.

The dosage form in accordance with the present invention is generally in the form of a tablet that is capable of readily dissolving and/or disintegrating upon contact with the oral mucosa, with saliva, or with other liquids present in the mouth of a patient. These dosage forms must include an effervescent couple and an adjusting substance.

Preferably, the dosage forms of the present invention have a surface area ranging from about 10=² to about 160 mm², more preferably ranging from about 15 mm² to about 100 mm², and most preferably ranging from about 18 mm² to about 72 mm². It is believed that as more dosage form surface area contacts damaged or irritated muscositis tissue, the greater the chance a clinically significant increase in fentanyl uptake will result.

Most preferably, the dosage forms of the present invention also include at least one effervescent couple and at least one pH adjusting substance. Any effervescent agent or effervescent couple may be used provided it is safe for human consumption. These include those described in U.S. Pat. Nos. 5,178,878, 5,503,846, 6,200,604, and U.S. patent application publication 2005/0163838A1 the texts of which are hereby incorporated by reference to the extent they discuss various effervescent couples, pH adjusting substances and constructions of dosage form including same. Effervescent couples generally are water or saliva activated materials usually kept in an anhydrous state with little or no absorbed moisture or in a stable hydrated form. Typically these involve at least one acid source and at least one source of a reactive base, usually a carbonate or bicarbonate.

The acids generally include food acids, acid anhydrides and acid salts. Food acids include citric acid, tartaric acid, malic acid, fumeric acid, adipic acid, ascorbic acid and succinic acid. Acid anhydrides or salts of these acids may be used. Salts in this context may include any known salt but in particular, sodium, dihydrogen phosphate, disodium dihydrogen phosphate, acid citrate salts and sodium acid sulfate. Bases useful in accordance with the invention typically include sodium bicarbonate, potassium bicarbonate and the like. Sodium carbonate, potassium carbonate, magnesium carbonate and the like may also be used to the extent they are used as part of an effervescent couple. However, they are more preferably used as a pH adjusting substance. Preferably, stoichiometric equivalent amounts of acid and base are used. It is possible, however, that some excess of acid, acid anhydrate, or acid salt or base be used. However, care should be exercised when so formulating a formulation particularly in view of the overall pH adjusting effect of such components, if any. An excess could affect absorption.

The amount of effervescent material useful in accordance with the present invention is an effective amount and is determined based on properties other than those which would be necessary to achieve disintegration of the tablet in the mouth. Instead, effervescence is used as a basis for enhancing transmission of the fentanyl across mucosal membranes via buccal, gingival or sublingual administration in the oral cavity. Accordingly, the amount of effervescent couple should range from between about 5 to about 85 percent, more preferably between about 15 to 60 percent, even more preferably between about 30 and 45 percent and most preferably between about 35 to about 40 percent, based on the weight of the total dosage form. Of course, the relative proportion of acid and base will depend upon the specific ingredients (for example, whether the acid is monoprotic, diprotic or triprotic) relative molecular weights, etc. However, preferably, a stoichiometric amount of each is provided although, of course, excesses are acceptable.

Preferably, formulations in accordance with the present invention include at least one pH adjusting substance. In this manner, a drug that is susceptible to changes in ionization state can be administered by affecting the proper conditions for its dissolution as well as transmission across one or more of the membranes or tissues within the oral cavity, for example, the oral mucosa. If the ideal conditions for transmission of a particular drug are basic, the addition of a sufficient excess of suitably strong acid as part of the manufacture of an effervescent couple or as a pH adjusting substance may not be indicated. The selection of another pH adjusting substance such as, for example, anhydrous sodium carbonate which operates separate and apart from the effervescent agents would be preferred.

pH adjusting substances in accordance with the present invention can be used to provide further permeation enhancement. The selection of the appropriate pH adjusting substance will depend on the drug to be administered and, in particular, to the pH at which it is ionized or unionized, and whether the ionized or unionized form facilitates transmission across the oral mucosa.

With regard to fentanyl and its salts, a basic substance is preferred to enhance delivery. pH adjusting substances in accordance with the present invention can include, without limitation, any substance capable of adjusting the localized pH to promote transport across the membranes in the oral cavity in amounts which will result in a pH's generally ranging from between about 5 to about 8 and more preferably between about 6 to about 7. The pH is the “localized pH” at the microenvironment in the mouth of a patient at the surface contact area of the oral mucosa and the dosage form or any portion thereof (such as when it disintegrates).

Preferably, the materials which can be used as pH adjusting substances include carbonates such as sodium, potassium or calcium carbonate or a phosphate such as calcium or sodium phosphate. Most preferred is sodium carbonate. The amount of pH adjusting substance presenting the dosage form can vary with the type of pH adjusting substance used, the amount of any excess acid or base from the effervescent couple, the nature of the remaining ingredients and, of course, the drug which, in this case, is fentanyl.

Most preferably the amount of pH adjusting substance will range from between about 0.5 to about 25 percent, more preferably between about 2 to about 20 percent, even more preferably between about 5 to about 15 percent and most preferably between about 7 to about 12 percent by weight based on the weight of the total dosage form. The most preferred pH adjusting substance is a carbonate, bicarbonate, or phosphate.

Also preferred are those pH adjusting substances which, when provided in a suitable amount, can provide a change in the localized pH of at least about 0.5 pH units, more preferably about 1.0 pH units and even more preferably about 2.0 pH units when compared to an otherwise identical formulation without the pH adjusting substance.

The dosage form may include other conventional excipients in generally known amounts to the extent they do not detract from the advantages described herein. These can include without limitation binders, sweeteners, coloring components, flavors, glidants, lubricants, preservatives, fillers, noneffervescent disintegrants, and the like.

Any filler or any amount of a filler may be used as long as the resulting dosage forms achieve the results described herein. Most preferred amongst the fillers are sugar and sugar alcohols and these may include nondirect compression and direct compression fillers. Nondirect compression fillers generally, at least when formulated, have flow and/or compression characteristics which make them impractical for use in high speed tableting process without augmentation or adjustment. For example, a formulation may not flow sufficiently well and therefore, a glidant such as, for example, silicon dioxide may need to be added.

Direct compression fillers, by contrast, do not require similar allowances. They generally have compressibility and flowability characteristics which allow them to be used directly. It is noted that, depending upon the method by which formulations are made, nondirect compression fillers may be imparted with the properties of direct compression fillers. The reverse is also true. As a general matter, non direct compression fillers tend to have a relatively smaller particle size when compared to direct compression fillers. However, certain fillers such as spray dried mannitol have relatively smaller particle sizes and yet are often directly compressible, depending upon how they are further processed. There are also relatively large nondirect compression fillers as well.

Fillers that are preferred in accordance with the present invention include mannitol, lactose, sorbitol, dextrose, sucrose, xylitol and glucose, to the extent their use can provide the results described herein. More preferably in accordance with the present invention, the filler is not lactose monohydrate used in an amount of 20% or more based on the weight of the formulation and even more preferably no lactose monohydrate is used. Most preferred in accordance with the present invention, spray dried mannitol is used. The amount of filler can range from 10 to about 80% and more preferably about 25 to about 80%, most preferably 35 to about 60% by weight of the formulation.

Noneffervescent disintegrants may also be used in accordance with the present invention. These may also include binders that have disintegrating properties. Disintegrants in accordance with the present invention can include microcrystalline cellulose, cross linked polyvinyl pyrrolidone (PVP-XL), sodium starch glycolate, croscarmellose sodium, cross-linked hydroxypropyl cellulose and the like. Of course, the selection of the disintegrant depends upon whether or not, in a given system, the results described herein may be obtained. More preferably, the formulation will be free of more than about 20% microcrystalline cellulose and cross linked polyvinyl pyrrolidone in an amount of about 5% or more, especially in a formulation that includes in additional 20% lactose monohydrate.

Most preferred for use as a disintegrant is a starch glycolate and in particular sodium starch glycolate. A particularly useful sodium starch glycolate is GLYCOLYS® (standard grade) available from Roquette of Lestrem France. Indeed, it is even more preferred that the formulation include neither microcrystalline cellulose nor cross-linked PVP.

The amount of noneffervescent disintegrant will vary with known factors such as, the size of the dosage form, the nature and amounts of the other ingredients used, etc. However, in general the amount should range from between about 0.25 to about 20% by weight of the final formulation, more preferably between about 0.5 to about 15% w/w, even more preferably 0.5 to about 10% w/w and even more preferably between about one and about eight percent by weight. This is again based on the weight of the finished formulation.

Also generally useful in accordance with the present invention is a tableting or ejection lubricant. The most common known lubricant is magnesium stearate and the use of magnesium stearate is preferred. Generally, the conventional wisdom behind tableting lubricants is that less is more. It is preferred in most circumstances that less than about one percent of a tableting lubricant be used. Typically, the amount should be half a percent or less. However, the amount of magnesium stearate used can be greater than 1.0%. Indeed, it is preferably greater than about 1.5% and most preferably about 1.5% and about 3%. Most preferred is the use of about 2% magnesium stearate. Other conventional tableting lubricants such as, for example, stearic acid, calcium stearate and the like may also be used in place of some or all of the magnesium stearate.

The oral dosage form is administered to alleviate pain including, but not limited to, cancer pain, breakthrough cancer pain, back pain, lower back pain, joint pain, pain from trauma or accidents, neuropathic pain, surgical or post-operative pain, any form of arthritic pain, muscle pain, or a pain from a disease or condition other than cancer.

To determine whether or not mucositis affects transmucosal administration of fentanyl, one need only undertake a routine human clinical study in a population of patients. The appropriate clinical study would use any of the traditional models. An example of such a study is as follows:

Clinical Study

A Phase 1, multicenter, open-label study was conducted in the US between April and September 2005. Each site obtained Institutional Review Board approval and all patients provided written informed consent. Following screening, enrolled patients were assigned to study groups on the basis of the presence or absence of active oral mucositis.

Male or female opioid-tolerant patients with cancer of 18 years or older were included in this study. Opioid tolerance was defined as having taken 60-1000 mg morphine/day or an equi-analgesic dose of another opioid for at least 1 week prior to the study. Patients with oral mucositis had to have grade 1-3 upon clinical examination and grade 1 or 2 upon functional/symptomatic examination using the Common Terminology Criteria for Adverse Events grading system. Patients with oral mucositis also had to agree to withhold topical treatment for oral mucositis and/or thrush between 1 hour before, and up to 8 hours after, FBT administration. In addition, patients had to have serum creatinine of at most 2.5 times the upper limit of normal (“ULN”), total bilirubin of at most 2.0 mg/dL, and of at most 3.0 times the ULN of alkaline phosphatase, aspartate aminotransferase and alanine aminotransferase.

Female patients were required to have a negative pregnancy test and were excluded if they had taken oral cyclical contraceptives within 2 weeks of the commencement of the study; other contraceptive measures were permitted. Patients were also excluded if one or more of the following criteria were present: active brain metastasis with raised intracranial pressure; chronic obstructive pulmonary disease with carbon dioxide retention; risk of significant bradyarrhythmia; hypersensitivity to fentanyl or FBT; use of inhibitors or inducers of cytochrome P450 3A4/5 isoforms, use of monoamine oxidase inhibitors within 2 weeks or fentanyl within 1 week prior to the start of the study; or any other condition likely to interfere with the conduct of the study.

In this particular study, fentanyl buccal tablet sold under the brand name FENTORA® and manufactured by Cephalon, Inc. was used to dose the patients. However, it should be understood that any oral dosage form comprising fentanyl, a pH adjusting substance, and an effervescent couple and capable of administration across the oral mucosa may be administered to provide similar results.

A single 200 μg dose of FBT was self-administered in the morning by placing the tablet between the upper gum and cheek above a molar tooth, allowing it to dissolve without disturbance for 10 minutes. Patients were instructed to gently massage the cheek for 5 minutes in the location of the tablet if it had not dissolved. Any remaining portion of tablet was swallowed with a glass (125 mL) of water after 30 minutes. In patients with mucositis, FBT was placed in an area of the mouth affected by mucositis.

For measurement of fentanyl concentrations, venous blood samples (4 ml) were collected immediately prior to, and 10, 20, 30, 40, 45, and 50 minutes and 1, 2, 3, 4, 6, and 8 hours following FBT administration. Samples were collected in tubes containing potassium ethylene diamine tetraacetic acid and inverted slowly 6-8 times to mix the contents before placing on water/ice (−4° C.). Plasma was harvested by centrifugation (1500 g, −15 min at 4° C.) within 5-60 min after collection. Samples were stored at −20° C. until assayed and each-sample was analyzed twice.

Concentrations of fentanyl were determined using a validated high-performance liquid chromatography method with tandem mass spectrometric detection (LC-MS/MS). A 100 μL aliquot of plasma was fortified with 25 μL internal standard (d₅ fentanyl) working solution. Analytes were isolated through liquid/liquid extraction using a hexane/methyl t-butyl ether/methylene chloride mixture. The resulting organic layer was evaporated to dryness and reconstituted using an appropriate solvent before injection into the LC-MS/MS apparatus. The quantifiable range of the fentanyl assay was 0.025 to 10.000 ng/mL. The lower limit of fentanyl quantitation was nominally 0.025 ng/mL. All samples from a given patient were analyzed in a single run.

The fentanyl pharmacokinetic parameters determined for each patient were: the absorption profile parameters of maximum plasma concentration (Cₘₐₓ) time to reach Cₘₐₓ (tₘₐₓ), and area under the plasma concentration-time curve from time zero to the median tₘₐₓ of patients without mucositis (AUC_{0-tmax′}); and AUC from time zero to 8 hours (AUC₀₋₈). Pharmacokinetic values were estimated by noncompartmental methods using WinNonlin® (Enterprise Version 4.1, Pharsight Corporation, Mountain View, Calif.).

Adverse events (AEs) were monitored throughout. Treatment-emergent AEs were defined as any AEs that began or worsened after drug administration. Measurements of vital signs were performed before, and at 10, 20, 30, 40, 45, and 50 minutes and 1, 2, 3, 4, 6, and 8 hours following, FBT administration.

Oral mucosal examinations were performed by the investigator to evaluate mucosal irritation prior to administration, at the end of the dwell time (defined as the time between tablet placement and its complete disappearance by visual inspection), and at 1, 2, 3, 4, and 8 hours following FBT administration. Oral mucosal examination findings in eight areas of the mouth were recorded at each time point. The eight areas were: maxillary labial mucosa; mandibular labial mucosa; right buccal mucosa; left buccal mucosa; right lateral and ventral tongue; left lateral and ventral tongue; floor of mouth and lingual frenum; and soft palate and fauces.

For the log-transformed values on each of Cₘₐₓ, AUC₀₋ₜₘₐₓ, and AUC₀₋₈, a 90% confidence interval (“CI”) for the difference of means was calculated, based on the two-sample t-test. The endpoints of the 90% CIs of these log-transformed variables were exponentiated to get 90% CIs for the ratio of means (i.e. patients with/patients without mucositis). A Wilcoxon rank sum test was performed on tₘₐₓ to compare the two populations.

Results

Demographics and Disposition of Subjects

Nineteen opioid-tolerant patients with cancer, 10 with and 9 without oral mucositis, were included in the study. Of these, 16 patients, 8 with and 8 without oral mucositis, received treatment with FBT and completed the study. Of the 3 patients withdrawn, 1 without mucositis experienced a severe AE before receiving study drug, and 2 with mucositis withdrew consent prior to treatment.

Patients had a mean age of 57.6 years; most were women. Since the number of patients in each group was small, no comparison of demographic characteristics was made. In the patients with mucositis, the clinical grade of mucositis was 1 for 8 patients, and the functional grade of mucositis was 1 for 7 patients and 2 for 1 patient.

Pharmacokinetic Assessments

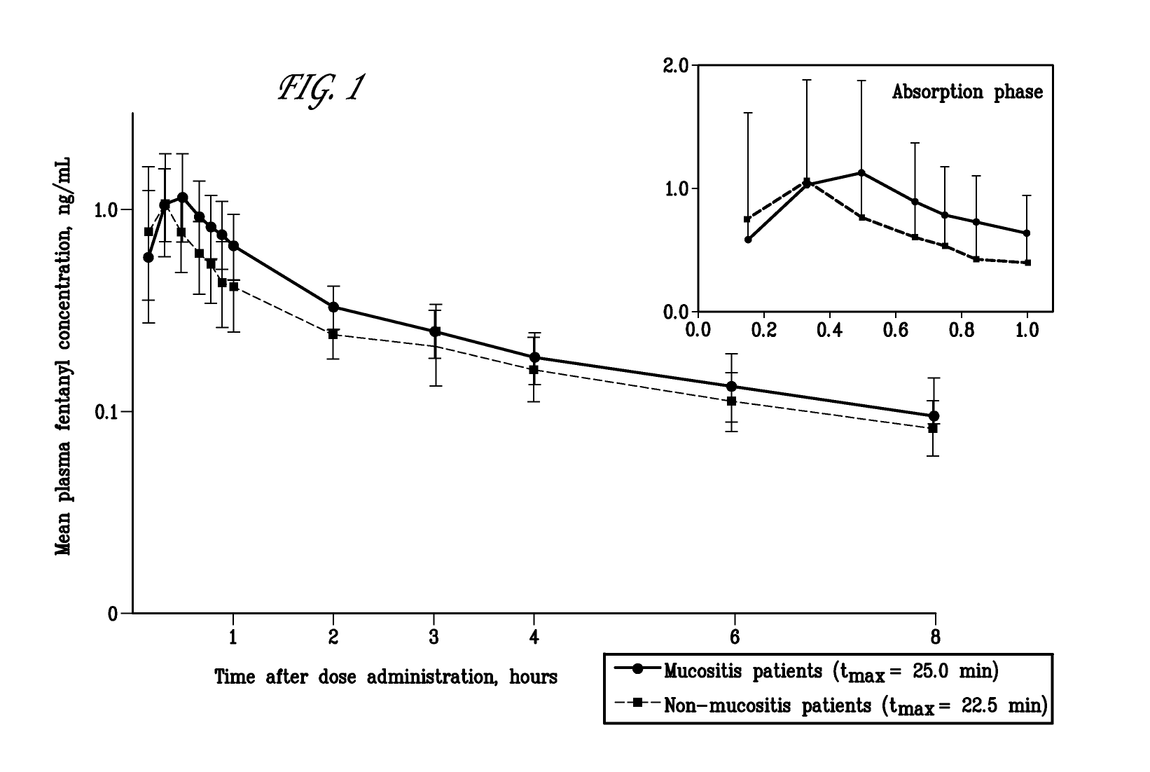

Pharmacokinetic parameters for FBT in the two groups are shown in the following table. Maximum plasma concentrations of fentanyl were achieved rapidly following FBT administration (FIG. 1) with a median tₘₐₓ of 25.0 min in patients with mucositis and 22.5 min in patients without mucositis (p=0.79). Mean Cₘₐₓ values were comparable with no statistically significant difference between the two patient populations: 1.25 ng/mL in patients with mucositis, and 1.24 ng/mL in patients without mucositis. Mean AUC₀₋ₜₘₐₓ, and AUC₀₋₈ values were 0.21 and 0.25 ng*hr/mL and 2.33 and 1.86 nghr/mL, respectively, for patients with and without mucositis.

The pharmacokinetics of FBT in opioid-tolerant cancer patients with and without mucositis is summarized in the Table 1 below.

TABLE 1
Pharmacokinetics of FBT in opioid-tolerant cancer
patients with and without mucositis

| | Patients | Patients |
|---|---|---|
| | with | without |
| | mucositis | mucositis |
| Parameter | (n = 8) | (n = 8) |
| Cₘₐₓ (ng/mL) | 1.25 ± 0.78 | 1.24 ± 0.77 |
| AUC₀₋₈ | 2.33 ± 0.93 | 1.86 ± 0.86 |
| (ng · h/mL) | | |
| AUC_{0-tmax′} | 0.21 ± 0.16 | 0.25 ± 0.24 |
| (ng · h/mL) | | |
| tₘₐₓ (min)* | 25.0 (15-45) | 22.5 (10.0-121.0) |

*Data are median (range), all other data are mean ± standard deviation.
**p-value based on a Wilcoxon rank sum test.

In total, 4 patients (1 patient with mucositis, 3 patients without) experienced at least 1 treatment-emergent AE during the study (table IV). Dizziness possibly or probably related to treatment was reported for 1 patient in each group; for both patients, the AE was mild in severity, and resolved. Anemia, nausea, and back pain (not treatment-related) were experienced by 1 patient each, all in the group without mucositis. All AEs were mild or moderate in severity. No cases of respiratory depression and no deaths were reported in the study. There were no serious AEs or withdrawals due to an AE. The patient prematurely withdrawn from the study due to a serious AE experienced intestinal obstruction of moderate severity before receiving any study drug. There were no clinically meaningful changes in vital signs in either group.

No application site AEs were reported, and no changes from baseline in individual oral mucosal assessments were noted at any assessment point after FBT administration. Seven of the 8 patients without mucositis were rated as normal and the one remaining patient without mucositis had no oral mucosal assessment at the beginning of the study. Patients with mucositis did have abnormal oral mucosal findings before and after FBT placement, but for each of the eight specific areas of the mouth examined, there were no changes in the number of abnormal findings reported over successive examinations.

In this study, the absorption profile of FBT was similar in cancer patients with and without mucositis. Maximum plasma fentanyl concentrations were achieved rapidly in both populations, suggesting that the relatively mild clinical and functional grades of mucositis had minimal effect on the absorption of active drug. Thus, FBT may be an appropriate treatment for BTP in opioid-tolerant cancer patients who also exhibit mild grades of oral mucositis.

Comparisons with the results in healthy volunteers obtained from other pharmacokinetic studies employing a 200 μg dose of FBT show that Cₘₐₓ was approximately 2-3 fold higher (1.25 vs. 0.4-0.6 ng/mL) and tₘₐₓ occurred earlier (22.5-25.0 vs. 40.2-45.6 min) in the present study. See Darwish M, Kirby M, Robertson P Jr. et al. Pharmacokinetic properties of fentanyl effervescent buccal tablets: a phase I, open-label, crossover study of single-dose 100, 200, 400, and 800 μg in healthy adult volunteers. Clin Ther 2006; 28: 707-14. See also Darwish M, Tempero K, Kirby M, Thompson J. Pharmacokinetics and dose proportionality of fentanyl effervescent buccal tablets in healthy volunteers. Clin Pharmacokinet 2005; 44: 1279-86. Between-study comparisons should be interpreted with caution, however, as differences in study populations and sampling techniques can impact the results. The current study enrolled patients with cancer on chronic opioid therapy, for example; previous pharmacokinetic studies have typically been conducted in healthy volunteers, with renal and hepatic function within the normal range as criteria for enrollment.

In conclusion, the results of this study show that the absorption profile of FBT is similar in patients with and without mild oral mucositis, suggesting there is no need to adjust the dose of FBT when mild oral mucositis is present. Further study is warranted, to extend these observations to higher doses of FBT in patients with more severe grades of mucositis.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating pain in a patient who also is afflicted with mild oral mucositis comprising: placing a fentanyl containing dosage form in intimate contact with the oral mucosa of said patient and retaining said dosage form in intimate contact with said oral mucosa for a time sufficient to allow uptake of at least a therapeutically significant amount of said fentanyl across said oral mucosa, said dosage form comprising an amount of said fentanyl of from about 50 μg to about 1400 μg based on the weight of the free base thereof, or an equivalent amount of a fentanyl salt, at least one pH adjusting substance, and at least one effervescent couple, said dosage from being designed to exhibit no clinically significant change in fentanyl uptake when dosed in patients having mild oral mucositis as compared with those patients not having mucositis.

2. The method of claim 1, wherein said amount of fentanyl ranges from about 100 μg to about 1250 μg based on the free base of said fentanyl, by weight of said dosage form.

3. The method of claim 2, wherein said amount ranges from about 100 μg to about 1000 μg based on the free base of said fentanyl, by weight of said dosage form.

4. The method of claim 1, wherein said at least one pH adjusting substance is selected and provided in an amount capable of providing a change in localized pH of at least 0.5 pH units.

5. The method of claim 4, wherein said pH adjusting substance is a carbonate, phosphate, or bicarbonate.

6. The method of claim 5, wherein said at least one pH adjusting substance is present in an amount of about 0.5% to about 25% by weight of said dosage form.

7. The method of claim 1, wherein said at least one effervescent couple is present in an amount of about 5% to about 85% by weight of said dosage form.

8. The method of claim 1, further comprising the steps of ingesting a liquid such that said patient's mouth becomes at least partially filled with said liquid and swallowing said liquid and any of said dosage form remaining in said mouth.

9. The method of claim 1, wherein said oral dosage form is placed between an upper gum and a cheek.

10. The method of claim 1, wherein said oral dosage form is placed under the tongue.

11. The method of claim 1, wherein said dosage form is maintained in contact with said oral mucosa for up to about 30 minutes.

12. The method of claim 11, wherein said dosage form is maintained in contact with said oral mucosa until said dosage form completely dissolves.

13. The method of claim 11, wherein said dosage form is maintained in contact with said oral mucosa, for between about 5 and about 30 minutes.

14. The method of claim 1, wherein said pain is selected from the group consisting of: cancer pain, breakthrough cancer pain, back pain, lower back pain, joint pain, pain from trauma, pain from accidents, neuropathic pain, surgical pain, post-operative pain, arthritic pain, muscle pain, and breakthrough pain from a disease or condition other than cancer.

15. The method of claim 1, wherein said dosage form further comprises one or more excipients.

16. The method of claim 15, wherein said one or more excipients are selected from the group consisting of binders, sweeteners, coloring components, flavors, glidants, lubricants, preservatives, fillers, disintegrants, and the like.

17. The method of claim 1, wherein dosing in said patients having mucositis increases fentanyl uptake by no more than about 15% as compared with fentanyl uptake in said patients not having mucositis.
